(19) United States
(12) Patent Application Publication
(10) Pub. No.: US 2016/0095698 A1
(43) Pub. Date: Apr. 7, 2016

(54) INTRAOCULAR LENS WITH FINE PATTERN

(71) Applicant: KOREA INSTITUTE OF SCIENCE AND TECHNOLOGY, Seoul (KR)

(72) Inventors: Hojeong JEON, Seoul (KR); Chunga KWON, Seoul (KR); Yu Chan KIM, Seoul (KR); Hyun Kwang SEOK, Seoul (KR); Jee-Wook LEE, Seoul (KR); Hyun Soo LEE, Seoul (KR); Choun Ki JOO, Seoul (KR)

(21) Appl. No.: 14/744,190

(22) Filed: Jun. 19, 2015

(30) Foreign Application Priority Data
Oct. 6, 2014 (KR) 10-2014-0134180

(51) Int. Cl. A61F 2/16

(52) U.S. Cl. CPC A61F 2/16; A61F 2002/1681

(57) ABSTRACT

Disclosed is an intraocular lens with a fine pattern including an optic having a circular shape, and a plurality of haptics connected to a periphery of the optic, and a pattern is formed on a surface of a partial area of at least one of the optic and the haptics.

### CROSS-REFERENCE TO RELATED APPLICATIONS

This application claims priority under 35 U.S.C. §119 to Korean Patent Application No. 10-2014-0134180 filed on Oct. 6, 2014 in the Korean Intellectual Property Office, the disclosure of which is incorporated herein by reference in its entirety.

### TECHNICAL FIELD

The present disclosure relates to an intraocular lens with a fine pattern, and more particularly, to an intraocular lens with a fine pattern, in which a pattern of an optimal shape for efficiently inhibiting the migration of lens epithelial cells is formed at an optimal location on the surface of the intraocular lens to dramatically reduce a migration rate of the corresponding cells, thereby effectively preventing a secondary cataract.

### BACKGROUND

A cataract is a thickening or clouding of the lens inside the eye, and is treated by various methods. Generally, to treat a cataract, a simple and safe surgical procedure to remove a clouded lens, and instead of the lens, insert an intraocular lens is applied, and it takes 15 minutes without anesthesia. As of 2011, according to the Korean National Health Insurance Service, it is reported that patients who had cataract surgery amounts to 308,000, and cataract surgery is known as one of the commonly performed surgical procedures.

Cataract surgery is a common and safe surgical procedure, but has a risk of complications such as a secondary cataract. The secondary cataract complication is a common complication appearing in about 50% of the total number of patients who underwent cataract surgery, and cells proliferate onto an intraocular lens and the lens capsule clouds up again.

Currently, a secondary cataract is treated with a Nd:YAG laser procedure, but the cost required for laser procedure is close to the cost of cataract surgery, and possible complications include, for example, damage of an intraocular lens, inflammation, an increase in intraocular pressure, and a retinal detachment phenomenon. Additionally, after the laser procedure, recently developed high-cost focusing adjustable IOLs would lose its original function. However, there is no technology for prevention of a common complication of secondary cataract after cataract surgery, causing blurry vision, and cataract surgery usually relies on laser treatment having side effects after surgery.

Therefore, there is the demand for development of technology to prevent a secondary cataract.

### RELATED LITERATURES

### Patent Literature

(Patent Literature 1) Korean Patent Registration No. 10-0314665

### SUMMARY

The present disclosure is designed to address the above issue, and therefore the present disclosure is directed to providing an intraocular lens with a fine pattern in which, dissimilar to a related art, an optimal pattern is formed on the surface of the intraocular lens to efficiently inhibit the migration of lens epithelial cells, thereby preventing a secondary cataract.

Also, the present disclosure is directed to providing an intraocular lens with a fine pattern in which an arrangement of a regular convex and concave region is implemented in an optimal shape to dramatically reduce about ⅕ or lower migration rate of lens epithelial cells that may lead to a secondary cataract.

Also, the present disclosure is directed to providing an intraocular lens with a fine pattern in which a location and a depth of the pattern on the intraocular lens, widths of a convex region and a concave region, and an angle of the pattern are optimized to effectively prevent a secondary cataract.

To achieve the above objects, a first embodiment of an intraocular lens with a fine pattern according to the present disclosure includes: an optic having a circular shape; and a plurality of haptics connected to a periphery of the optic, wherein a pattern is formed on a surface of a partial area of at least one of the optic and the haptics.

The pattern may have a concave-convex shape including a concave region and a convex region, the convex region may have at least one shape of a stripe, a cone, a hemisphere, a square, and a quadrangular pyramid, and the concave region may have a first arrangement representing an arrangement in one direction and the other direction intersecting each other or a second arrangement representing an arrangement in one direction.

A width of the convex region may be between 0.2 and 4% of a thickness of the haptics, a width of the concave region may be between 0.4 and 5% of a thickness of the haptics, and a thickness of the haptics may be between 300 and 500 μm.

Also, a width of the convex region may be between 1 and 12 μm, a width of the concave region may be between 2 and 15 μm, and the pattern may be formed on at least one of a front area, a rear area, and a side area of at least one of the optic and the haptics.

The pattern formed on the side area of the optic may be formed at an angle between 80 and 100° with respect to an optical axis of the optic, the pattern formed on at least one of the haptics and at least one of the front area and the rear area of the optic may be formed at an angle between −10 and 10° with respect to the optical axis of the optic, and the pattern formed on the haptics may be formed on an area of the haptics adjacent to the optic.

Also, the pattern may be formed on at least one of a front outer circumferential area, a rear outer circumferential area, and a side area of the optic, a width of at least one of the front outer circumferential area and the rear outer circumferential area may be between 5 and 14% of a diameter of the optic, and a width of the side area may be between 25 and 40% of the thickness of the haptics.

Also, the width of at least one of the front outer circumferential area and the rear outer circumferential area may be between 400 and 700 μm, the width of the side area may be between 80 and 160 μm, and a diameter of the optic may be between 5000 and 8000 μm.

A depth of the pattern may be between 0.6 and 3% of a thickness of the haptics, and a depth of the pattern may be between 3 and 9 μm.

Subsequently, a second embodiment of an intraocular lens with a fine pattern according to the present disclosure includes: an optic having a circular shape; and a plurality of haptics connected to a periphery of the optic, wherein a pattern of a concave-convex shape including a concave region and a convex region is formed on a surface of a partial area of at least one of the optic and the haptics, and a width of the convex region is smaller than or equal to a width of the concave region.

A ratio of the width of the convex region and the width of the concave region may be between 1:1 and 1:1.7, the width of the convex region may be between 3 and 5 μm, and the width of the concave region may be 5 μm.

Also, the convex region may have at least one shape of a stripe, a cone, a hemisphere, a square, and a quadrangular pyramid, and the concave region may have a first arrangement representing an arrangement in one direction and the other direction intersecting each other or a second arrangement representing an arrangement in one direction.

According to the intraocular lens with a fine pattern of the present disclosure, dissimilar to a related art, there is an advantage of efficiently inhibiting the migration of lens epithelial cells to prevent a secondary cataract, by forming an optimal pattern on the surface of the intraocular lens.

Also, there is an advantage of dramatically reducing about ⅕ or lower migration rate of lens epithelial cells that may lead to a secondary cataract, by implementing an arrangement of the convex region and the concave region in an optimal shape for the shapes and behavioral characteristics of lens epithelial cells.

Also, there is an advantage of effectively preventing a secondary cataract, by optimizing a location and a depth of the pattern on the intraocular lens, widths of the convex region and the concave region, and an angle of the pattern.

### BRIEF DESCRIPTION OF THE DRAWINGS

FIGS. 1A-1B show a plane view and a side view of an intraocular lens with a fine pattern according to the present disclosure.

FIGS. 2A-2B show a plane view and a side view of an intraocular lens with a fine pattern according to the present disclosure.

FIGS. 3A-3C are photographic images of an intraocular lens with a fine pattern according to the present disclosure.

FIG. 4 is a diagram illustrating a concave region and a convex region of a pattern of the present disclosure.

FIGS. 5A-5C are photographic images illustrating patterns of various shapes formed according to the present disclosure.

FIGS. 6A-6D chronologically illustrate a general method of fabricating a pattern to verify the cell migration inhibitory effect of a fine pattern.

FIG. 7 is a photographic image illustrating migration of lens epithelial cells in a patterned substrate according to the present disclosure over time.

FIG. 8 is a photographic image illustrating migration of lens epithelial cells in a patterned substrate according to the present disclosure over time.

FIG. 9 is a graph illustrating a cell migration inhibitory effect based on widths of a concave region and a convex region of a pattern of an intraocular lens with a fine pattern according to the present disclosure.

FIG. 10 is a graph illustrating a cell migration inhibitory effect based on widths of a concave region and a convex region of a pattern of a patterned substrate according to the present disclosure.

FIG. 11 is a diagram illustrating behaviors and migration of lens epithelial cells in a patterned substrate according to the present disclosure.

### DETAILED DESCRIPTION OF EMBODIMENTS

Hereinafter, preferred embodiments of an intraocular lens with a fine pattern according to the present disclosure are described in detail with reference to the accompanying drawings. The present disclosure will become apparent from the following description of the embodiments, and these embodiments are provided for illustrative purposes only and are not intended to limit the scope of protection defined by the appended claims.

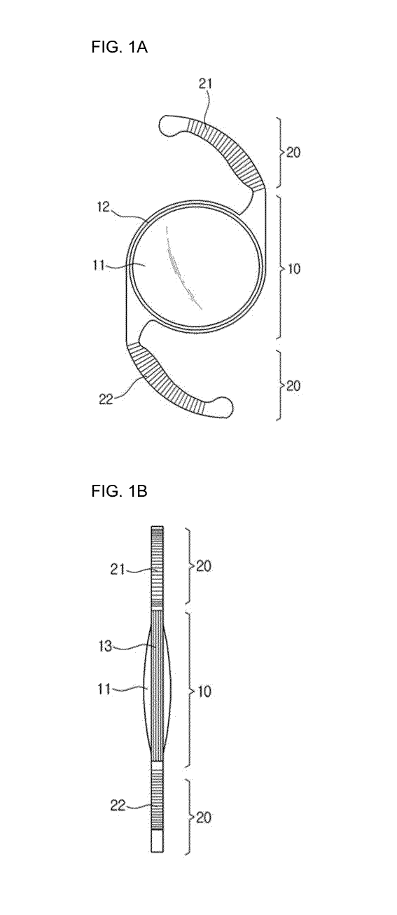

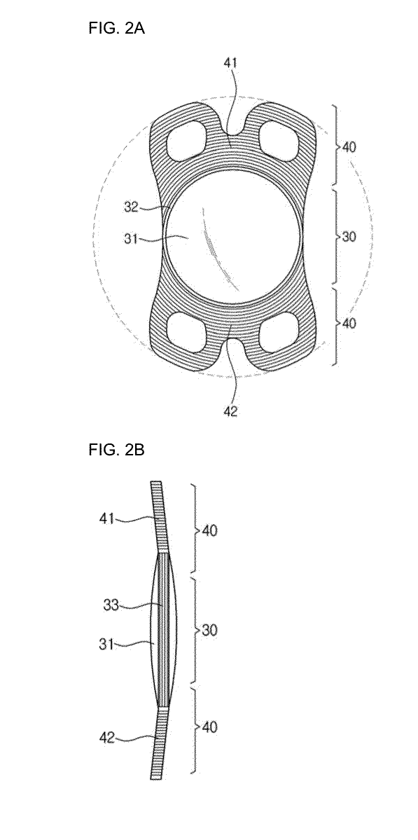

A first embodiment of an intraocular lens with a fine pattern according to the present disclosure includes an optic 10 and 30; and haptics 20 and 40, as shown in FIGS. 1A-1B and 2A-2B.

First, the optic 10 refers to a part of a circular shape serving as a lens of an intraocular lens.

Also, the haptic 20 refers to a part connected to the periphery of the optic 10 and 30, and serves as a support. The haptics 20 and 40 may, as shown in FIGS. 1A-1B and 2A-2B, have various shapes, but because at least one haptic should be provided at each side of the optic 10 and 30, multiple haptics are preferred, and more preferably, two to four haptics are effective.

The optic 10 and 30 and the haptics 20 and 40 may be made of various transparent materials, but the optic 10 and 30 and the haptics 20 and 40 are preferably made of at least one of plastic, silicone, and acryl, more preferably acrylic resin, most preferably polymethylmethacrylate resin and poly(HEMA) resin, which are effective in implementing an excellent lens function and efficiently forming and functionalizing the pattern of the present disclosure.

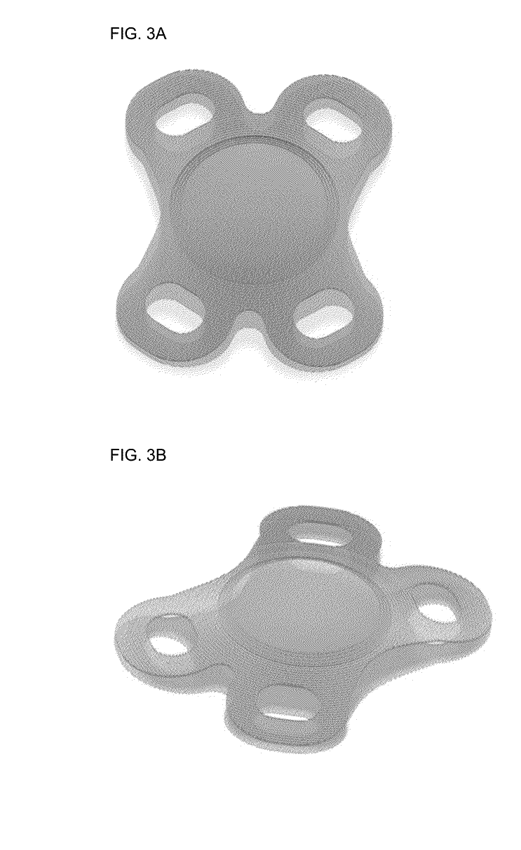

Also, as shown in FIGS. 3A-3C, it is preferred to form the pattern on the surface of a partial area of at least one of the optic 10 and 30 and the haptics 20 and 40. To prevent lens epithelial cells that may lead to a secondary cataract from proliferating and migrating onto the optic of intraocular lens, forming a pattern on the intraocular lens is effective.

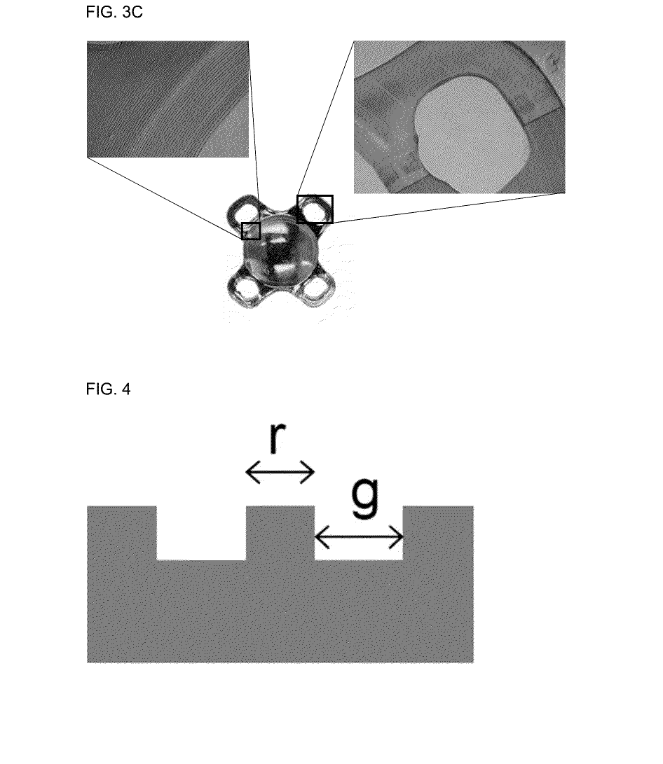

Here, patterns 12, 13, 21, 22, 32, 33, 41, and 42 may have any shape that enables the inhibition of migration of lens epithelial cells, but as shown in FIG. 4, a concave-convex shape including a concave region (g) and a convex region (r) is the most effective.

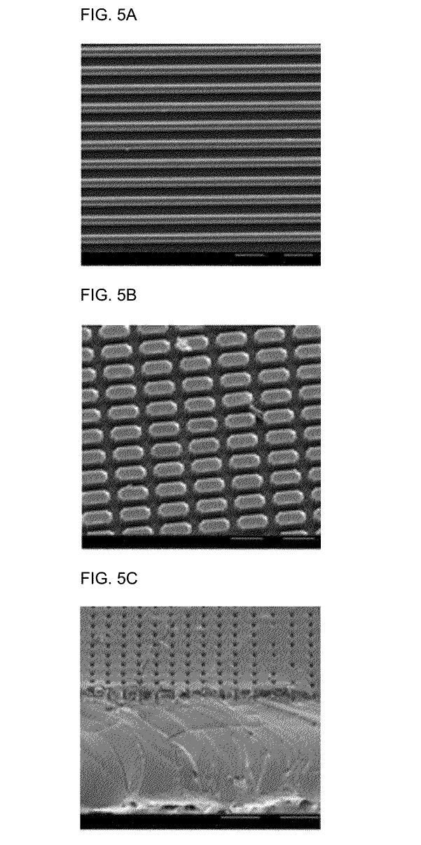

Also, as shown in FIGS. 5A-5C, the convex region preferably has at least one shape of a stripe, a cone, a hemisphere, a square, and a quadrangular pyramid, and by virtue of this shape, grooves/ridges, islands or holes are found as shown in FIGS. 5A-5C.

A width of the convex region is preferably between 0.2 and 4% of a thickness of the haptics 20 and 40, and it is effective that the width of the convex region is more preferably between 0.5 and 2% of the thickness of the haptics 20 and 40, most preferably between 0.6 and 1.6%. When the width of the convex region is less than 0.2% or more than 4%, there is a problem with the growth and migration of lens epithelial cells on the convex region or in the concave region due to the size and behavioral characteristics of the cells.

Also, the width of the convex region is preferably between 1 and 12 μm, and it is effective that the width of the convex region is more preferably between 2 and 6 μm, most preferably between 3 and 5 μm, and within this range, the cell growth and migration may be effectively inhibited.

Also, the concave region preferably has a first arrangement representing an arrangement in one direction and the other direction intersecting each other as shown in FIGS. 5A-5C; or a second arrangement representing an arrangement in one direction as shown in FIG. 5A. This forms an optimal pattern shape for lens epithelial cells, along with the shape of the convex region.

Also, a width of the concave region is preferably between 0.4 and 5% of the thickness of the haptics 20 and 40, and it is effective that the width of the concave region is more preferably between 0.8 and 2.3% of the thickness of the haptics 20 and 40, most preferably between 1 and 1.6%. When the width of the concave region is less than 0.4% or more than 5%, there is a problem with cells readily grown and migrated in the concave region due to the size and behavioral characteristics of lens epithelial cells.

Also, the width of the concave region is preferably between 2 and 15 μm, and it is effective that the width of the concave region is more preferably between 4 and 7 μm, most preferably 5 μm, and within this range, the cell growth and migration may be effectively inhibited.

Also, the thickness of the haptics 20 and 40 is preferably between 200 and 500 μm, and it is effective that the thickness of the haptics 20 and 40 is more preferably between 300 and 500 μm, most preferably between 300 and 430 μm. When the thickness of the haptics 20 and 40 is less than 200 μm, it is difficult to perform a support function when considering a thickness of the optic 10 and 30 for functioning as a lens and it is impossible to implement an optimal ratio when considering the size and width of the pattern, and when the thickness of the haptics 20 and 40 is more than 500 μm, there is a high possibility that cells migrate at an area other than the pattern due to the excessive thickness, and it is impossible to implement an optimal ratio between the width and the size of the pattern.

Also, the pattern is preferably formed on at least one of a front area, a rear area, and a side area of at least one of the optic 10 and 30 and the haptics 20 and 40, and it is effective that the pattern is more preferably formed on the rear area of the haptics.

The patterns 21, 22, 41, and 42 on the haptics 20 and 40 may be generated over the entire haptic 20 and 40 or on any area of the haptics 20 and 40, but are preferably formed on an area of the haptics 20 and 40 adjacent to the optic 10 and 30. That is, when the pattern is formed from an area of the haptics 20 and 40 adjacent to the optic 10 and 30 to a central area of the haptics 20 and 40, it is effective in inhibiting the migration of lens epithelial cells.

Also, for the pattern 13 and 33 formed on the side area of the optic 10 and 30, it is preferred to form the pattern 13 and 33 at an angle between 80 and 100° with respect to an optical axis of the optic 10 and 30, and it is effective to form the pattern 13 and 33 more preferably at an angle between 85 and 95°, most preferably an angle of 90°. On the side area of the optic 10 and 30, forming the pattern in the direction perpendicular to the optical axis of the optic 10 and 30, i.e., the lens, is the most effective in preventing the migration of lens epithelial cells in the corresponding direction, while not interrupting the function of the optic 10 and 30, dissimilar to the other areas.

For the pattern formed on the areas other than the side area of the optic 10 and 30, that is, at least one of the haptics 20 and 40 and at least one of the front area and the rear area of the optic 10 and 30, it is preferred to form the pattern at an angle between −10 and 10° with respect to the optical axis of the optic 10 and 30, and it is effective to form the pattern more preferably at an angle between −5 and 5°, most preferably an angle of 0°. On the areas other than the side area of the optic 10 and 30, forming the pattern in the direction parallel to the optical axis of the optic 10 and 30, i.e., the lens, is the most effective in inhibiting the migration of lens epithelial cells in the corresponding direction, dissimilar to the other areas.

Also, the pattern is preferably formed on at least one of the front outer circumferential area 12 and 32, the rear outer circumferential area 12 and 32, and the side area 13 and 33 of the optic 10 and 30. This is a structure for efficiently inhibiting the migration of cells into the optic 10 and 30 while maintaining the lens function of the optic 10 and 30.

A width of at least one of the front outer circumferential area 12 and 32 and the rear outer circumferential area 12 and 32 is preferably between 5 and 14% of a diameter of the optic 10 and 30, and it is effective that the width of at least one of the front outer circumferential area 12 and 32 and the rear outer circumferential area 12 and 32 is more preferably between 6 and 12% of the diameter of the optic 10 and 30, most preferably between 6.8 and 11%. When the width is less than 5%, the inhibitory effect of cells migrating onto the center of the front area or the rear area of the optic 10 and 30 is significantly reduced, and when the width is more than 14%, the lens function of the optic 10 and 30 is rather reduced due to the pattern.

Also, the width of at least one of the front outer circumferential area 12 and 32 and the rear outer circumferential area 12 and 32 is preferably between 400 and 700 μm, and it is effective that the width of at least one of the front outer circumferential area 12 and 32 and the rear outer circumferential area 12 and 32 is more preferably between 480 and 600 μm, most preferably 550 μm. Within this range, the cell migration into the lens may be effectively inhibited without a reduction in the lens function.

A width of the side area 13 and 33 of the pattern is preferably between 25 and 40% of the thickness of the haptics 20 and 40, and it is effective that the width of the side area 13 and 33 is more preferably between 30 and 35% of the thickness of the haptics 20 and 40, most preferably 33%. Within this range, the migration of lens epithelial cells onto the side area may be efficiently prevented, while maintaining the function of the lens.

Also, the width of the side area 13 and 33 is preferably between 80 and 160 μm, and it is effective that the width of the side area 13 and 33 is more preferably between 90 and 120 μm, most preferably 110 μm. When considering the thickness of the haptics 20 and 40, this width range is optimal for cell migration inhibition.

Also, a diameter of the optic 10 and 30 is preferably between 5000 and 8000 μm, and it is effective that the diameter of the optic 10 and 30 is more preferably between 5500 and 6000 μm, most preferably 5800 μm. This is an optimum diameter range for implementing the lens function when considering the size of the haptics 20 and 40 and the width and depth of the pattern.

A depth of the pattern is preferably between 0.6 and 3% of the thickness of the haptics 20 and 40, and it is effective that the depth of the pattern is more preferably between 0.9 and 1.5% of the thickness of the haptics 20 and 40, most preferably 1.2%. When the depth of the pattern is less than 0.6%, the cell inhibitory effect of the pattern is insufficient, and when the depth of the pattern is more than 3%, there is a problem with reduced durability of the pattern and cell migration in the vertical direction based on the width of the concave region.

Also, the depth of the pattern is preferably between 3 and 9 μm, and it is effective that the depth of the pattern is more preferably between 4 and 7 μm, most preferably 5 μm. Within this range, the cell inhibitory effect of the pattern is maximized.

A second embodiment of an intraocular lens with a fine pattern according to the present disclosure includes, as shown in FIGS. 1A-1B and 2A-2B, an optic 10 and 30 having a circular shape; and a plurality of haptics 20 and 40 connected to the periphery of the optic.

It is preferred to form a concave-convex pattern including a concave region and a convex region on the surface of a partial area of at least one of the optic 10 and 30 and the haptics 20 and 40, and it is effective that a width of the convex region is smaller than or equal to a width of the concave region. As a result of several tests, the inventor discovered that when the width of the convex region was smaller than equal to the width of concave region, a migration rate of lens epithelial cells tended to dramatically reduce.

Specifically, a ratio of the width of the convex region and the width of the concave region is preferably between 1:1 and 1:1.7, and it is effective that the ratio of the width of the convex region and the width of the concave region is more preferably between 1:1 and 1:1.66. Within this range, it was found that a migration inhibitory rate of lens epithelial cells dramatically increased due to the pattern formation.

Also, the width of the convex region between 3 and 5 μm and the width of the concave region of 5 μm is the most effective in preventing a secondary cataract.

Also, similar to the first embodiment of the present disclosure, preferably, the convex region has at least one shape of a stripe, a cone, a hemisphere, a square, and a quadrangular pyramid, and the concave region has a first arrangement representing an arrangement in one direction and the other direction intersecting each other; or a second arrangement representing an arrangement in one direction.

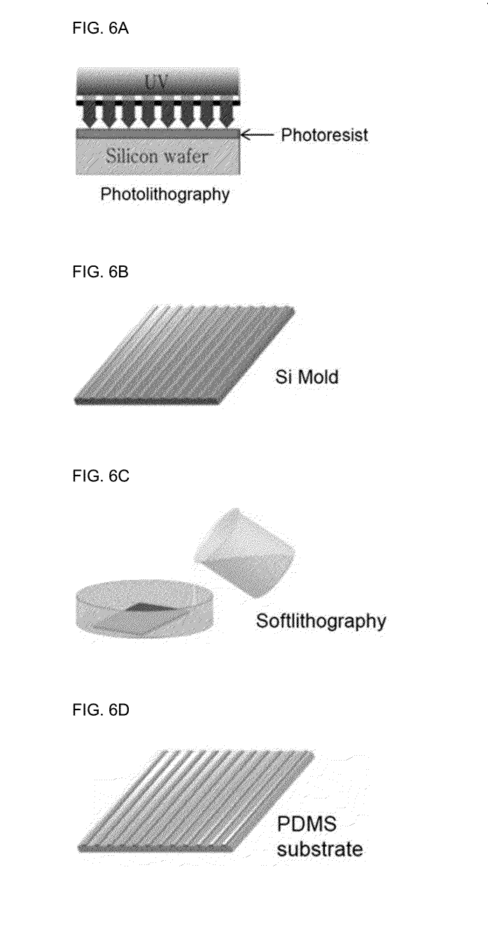

The intraocular lens with a fine pattern according to the present disclosure may be manufactured by any technique which enables pattern formation, and as shown in FIGS. 6A-6D, the intraocular lens with a fine pattern may be manufactured by photolithographic technique, and may be manufactured by laser technique, for example, femtosecond laser ablation.

A test was conducted to demonstrate the excellence of the intraocular lens with a fine pattern according to the present disclosure, and its result is described as below.

First, as shown in FIGS. 3A-3C, it can be seen that the intraocular lens manufactured by the present disclosure includes an optic and haptics and a pattern is formed on the optic and haptic, and as shown in FIGS. 5A-5C, it can be seen that patterns of various shapes may be formed.

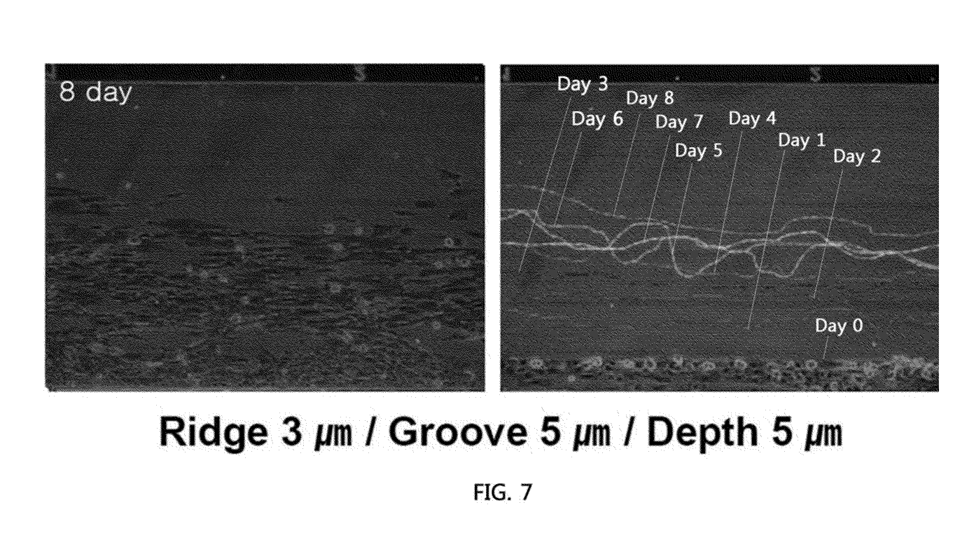

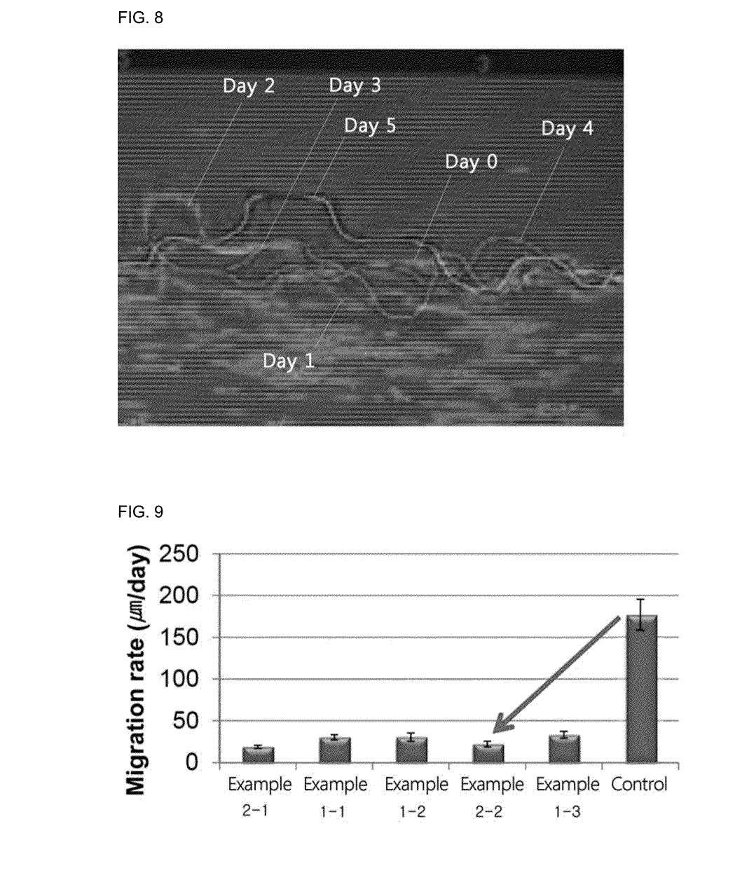

Also, FIGS. 7 and 8 are photographic images obtained by taking pictures of lens epithelial cells migrating onto an intraocular lens with a pattern 5 μm deep including a convex region 3 μm wide and a concave region 5 μm wide over time, and show an extent of migration of cells over time.

In this way, a test was each conducted on a general polymer substrate with no pattern as a comparative example (control) and Examples 1 and 2 of the present disclosure, as seen in the following Table 1. Examples 1-1, 1-2, and 1-3 indicate a pattern corresponding to only Example 1 of the present disclosure, and Examples 2-1 and 2-2 are also included in Example 1 of the present disclosure but are separately labeled as Example 2.

TABLE 1

| | Exam- | Exam- | Exam- | Exam- | Exam- | Con- |
|---|---|---|---|---|---|---|
| | ple 1-1 | ple 1-2 | ple 1-3 | ple 2-1 | ple 2-2 | trol |
| Width of convex | 10 | 5 | 5 | 3 | 5 | 0 |
| region(μm) | | | | | | |
| Width of concave | 5 | 3 | 10 | 5 | 5 | 0 |
| region(μm) | | | | | | |

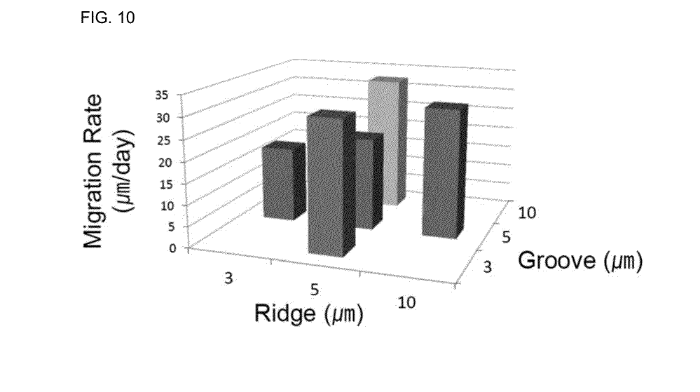

As shown in FIGS. 9 and 10, the experimental results demonstrated that all the examples exhibited a remarkable reduction in a cell migration rate up to 500% or higher in comparison to the control with no pattern, and particularly, it was found that Examples 2-1 and 2-2 had a superior cell migration inhibitory effect up to 200% or higher when compared to Examples 1-1, 1-2, and 1-3.

That is, this test proved that the pattern of the present disclosure had an excellent migration inhibitory effect of lens epithelial cells, and because an additional inhibitory effect of 200% or higher can be produced based on the width of the convex region and the concave region of the pattern, critical significance of the size of the pattern was also verified.

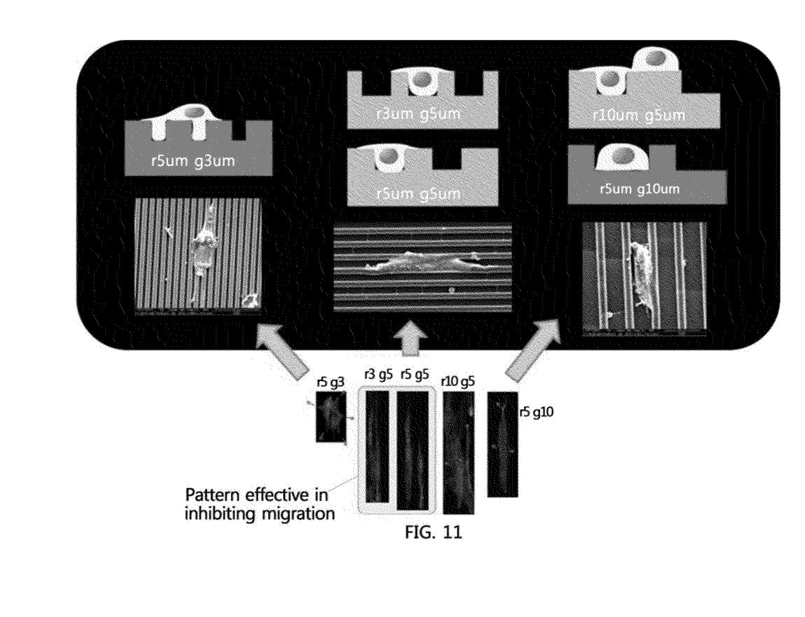

The migration inhibitory effect of lens epithelial cells of the pattern results from unique size and behavioral characteristics of lens epithelial cells as shown in FIG. 11, and the present disclosure optimized for such characteristics may maximize the effect.

While the preferred embodiments of the present disclosure have been described, various changes and equivalents may be made to the present disclosure. It will be apparent that the present disclosure may be properly modified and equally applied. Therefore, the above disclosure is not intended to limit the scope of the present disclosure as defined in the following claims.

What is claimed is:

1. An intraocular lens with a fine pattern, comprising:
an optic having a circular shape; and
a plurality of haptics connected to a periphery of the optic,
wherein a pattern is formed on a surface of a partial area of at least one of the optic and the haptics.

2. The intraocular lens with a fine pattern according to claim 1, wherein the pattern has a concave-convex shape including a concave region and a convex region.

3. The intraocular lens with a fine pattern according to claim 2, wherein the convex region has at least one shape of a stripe, a cone, a hemisphere, a square, and a quadrangular pyramid.

4. The intraocular lens with a fine pattern according to claim 2, wherein the concave region has a first arrangement representing an arrangement in one direction and the other direction intersecting each other or a second arrangement representing an arrangement in one direction.

5. The intraocular lens with a fine pattern according to claim 2, wherein a width of the convex region is between 0.2 and 4% of a thickness of the haptics.

6. The intraocular lens with a fine pattern according to claim 2, wherein a width of the concave region is between 0.4 and 5% of a thickness of the haptics.

7. The intraocular lens with a fine pattern according to claim 2, wherein a thickness of the haptics is between 200 and 500 μm.

8. The intraocular lens with a fine pattern according to claim 2, wherein a width of the convex region is between 1 and 12 μm.

9. The intraocular lens with a fine pattern according to claim 2, wherein a width of the concave region is between 2 and 15 μm.

10. The intraocular lens with a fine pattern according to claim 1, wherein the pattern is formed on at least one of a front area, a rear area, and a side area of at least one of the optic and the haptics.

11. The intraocular lens with a fine pattern according to claim 10, wherein the pattern formed on the side area of the optic is formed at an angle between 80 and 100° with respect to an optical axis of the optic.

12. The intraocular lens with a fine pattern according to claim 10, wherein the pattern formed on at least one of the haptics and at least one of the front area and the rear area of the optic is formed at an angle between −10 and 10° with respect to the optical axis of the optic.

13. The intraocular lens with a fine pattern according to claim 10, wherein the pattern formed on the haptics is formed on an area of the haptics adjacent to the optic.

14. The intraocular lens with a fine pattern according to claim 10, wherein the pattern is formed on at least one of a front outer circumferential area, a rear outer circumferential area, and a side area of the optic.

15. The intraocular lens with a fine pattern according to claim 14, wherein a width of at least one of the front outer circumferential area and the rear outer circumferential area is between 5 and 14% of a diameter of the optic.

16. The intraocular lens with a fine pattern according to claim 14, wherein a width of the side area is between 25 and 40% of the thickness of the haptics.

17. The intraocular lens with a fine pattern according to claim 14, wherein the width of at least one of the front outer circumferential area and the rear outer circumferential area is between 400 and 700 μm.

18. The intraocular lens with a fine pattern according to claim 14, wherein the width of the side area is between 80 and 160 μm.

19. The intraocular lens with a fine pattern according to claim 1, wherein a diameter of the optic is between 5000 and 8000 μm.

20. The intraocular lens with a fine pattern according to claim 1, wherein a depth of the pattern is between 0.6 and 3% of a thickness of the haptics.

21. The intraocular lens with a fine pattern according to claim 1, wherein a depth of the pattern is between 3 and 9 μm.

22. An intraocular lens with a fine pattern, comprising:
an optic having a circular shape; and
a plurality of haptics connected to a periphery of the optic,
wherein a pattern of a concave-convex shape including a concave region and a convex region is formed on a surface of a partial area of at least one of the optic and the haptics, and
a width of the convex region is smaller than or equal to a width of the concave region.

23. The intraocular lens with a fine pattern according to claim 22, wherein a ratio of the width of the convex region and the width of the concave region is between 1:1 and 1:1.7.

24. The intraocular lens with a fine pattern according to claim 22, wherein the width of the convex region is between 3 and 5 μm, and the width of the concave region is 5 μm.

25. The intraocular lens with a fine pattern according to claim 22, wherein the convex region has at least one shape of a stripe, a cone, a hemisphere, a square, and a quadrangular pyramid, and the concave region has a first arrangement representing an arrangement in one direction and the other direction intersecting each other or a second arrangement representing an arrangement in one direction.
